(19) United States
(12) Patent Application Publication
(10) Pub. No.: US 2011/0137293 A1
(43) Pub. Date: Jun. 9, 2011

(54) DISTAL PART GUIDING CATHETER

(75) Inventors: Deok Hee Lee, Seoul (KR); Seon Moon Hwang, Gyeonggi-do (KR)

(73) Assignee: THE ASAN FOUNDATION, Seoul (KR)

(21) Appl. No.: 13/058,317

(22) PCT Filed: Aug. 13, 2009
(86) PCT No.: PCT/KR2009/004538

§ 371 Date: Feb. 9, 2011

(30) Foreign Application Priority Data
Aug. 13, 2008 (KR) 20-2008-0010804

(51) Int. Cl. A61M 25/00

(52) U.S. Cl. 604/523

(57) ABSTRACT

Provided is a guiding catheter inserted into the closest part of a target vessel to easily perform various operations when an intervention is operated in the vessel, and including a circular hole for blood circulation to secure normal blood flow of a target organ when a blood flow occlusion phenomenon due to reduction in inner diameter of the vessel occurs.

### TECHNICAL FIELD

The present invention relates to a guiding catheter.

### BACKGROUND ART

A guiding catheter is used in various interventions for conveying a treatment devices to a treatment portion through a body passage such as a blood vessel.

An ideal guiding catheter must have the following characteristics: the guiding catheter must be inserted into a target vessel in a stable state, and sufficiently secure an internal cavity to easily introduce and remove various catheter and surgical instruments. In addition, when various instruments such as a stent, a balloon angioplasty catheter, etc. are inserted, the guiding catheter must be securely maintained in the vessel without any position change, and the guiding catheter must not cause vasospasm or damage to the vessel.

However, the guiding catheter that is currently on the market has several problems. In particular, in the case of a cerebrovascular or coronary intervention, since finer operations to a distal part are currently needed, there are several problems that cannot be easily solved by the current guiding catheter only. The problems of the guiding catheter that is currently on the market are as follows:

1. While a distal end of the guiding catheter is too rigid such that original characteristics of the guiding catheter can be maintained, vasospasm occurs frequently, and guidance to the far distal vessels is difficult especially when the vessels have smaller diameter and/or tortuous.

2. In the case of a tortuous vessel, guidance of the distal part is impossible. In addition, when the guiding catheter goes through a tortuous part, the vessel may be forcedly kinked to cause temporary occlusion of the vessel.

3. The smallest outer diameter of an available guiding catheter is 5 Fr. When the guiding catheter is inserted into a vessel having a small inner diameter such as a vertebral artery, etc., spasm, occlusion or damage to the vessel may occur.

4. When a stent is inserted into the intracranial vessels, in addition to being securely guided, there are many cases where a guiding catheter is guided beyond a generally-guided position, but this may be impossible in some patients. 5. When the vessel has a small diameter, insertion of the guiding catheter may cause occlusion of normal blood flow.

### DISCLOSURE

### [Technical Problem]

In order to solve the foregoing and/or other problems, it is an object of the present invention to provide a guiding catheter capable of easily performing various operations in a vessel, and inserting a catheter to the closest part of a target vessel when an intervention in the vessel is performed.

### [Technical Solution]

The foregoing and/or other aspects of the present invention may be achieved by providing a guiding catheter in which a blood flow circulation hole is formed in a side surface of a catheter to maintain normal blood flow of a target organ when the distal segment of a guiding catheter causes occlusion of blood flow.

In addition, the number of circulation holes may be at least one depending on necessity, and the size and shape of the blood flow circulation hole are not particularly limited as long as blood flow can be normally maintained.

Further, the catheter may have a minimized thickness while having a maximized inner diameter to provide a sufficient lumen to perform operations in a vessel and having sufficient flexibility for easy adaptation to the tortuous vessels.

### ADVANTAGEOUS EFFECTS

According to the present invention, it is possible to provide a guiding catheter having a large inner diameter capable of easily performing various operations in a vessel, and inserting a catheter to the closest part of a target vessel when an intervention in the vessel which can be tortuous is performed.

### DESCRIPTION OF DRAWINGS

The above and other aspects and advantages of the present invention will become apparent and more readily appreciated from the following description of exemplary embodiments, taken in conjunction with the accompanying drawings of which:

FIG. 1 is a view showing a state in which a conventional guiding catheter is inserted into a vessel of an internal carotid artery, and an angiogram thereof;

FIG. 2 is a view showing a state in which the conventional guiding catheter is inserted into a vessel of a vertebral artery, and an angiogram thereof;

FIG. 3 is angiograms showing states in which the conventional guiding catheters are inserted into tortuous vessels;

FIG. 4 is angiograms showing states in which spasm (elliptical dotted lines) of vessels are generated by the conventional guiding catheters;

FIG. 5 shows a far-reaching guiding catheter in accordance with an exemplary embodiment of the present invention; and

FIG. 6 shows a state in which the distal part of the far-reaching guiding catheter in accordance with the exemplary embodiment of the present invention is inserted into the closest part of a target vessel for treatment.

### DESCRIPTION OF MAJOR REFERENCE NUMERALS

| | |
|---|---|
| 10: Brachiocephalic artery | 20: Common carotid artery |
| 30: Subclavian artery | 40: Vertebral artery |
| 50: Basilar Artery | 60: External carotid artery |

70: Internal carotid artery
100: Distal end of conventional guiding catheter
110: Distal part: IV-V
111: Side surface hole for maintaining blood flow
112: Tip of guiding catheter
113: Cross-sectional view taken along dotted lines

| | |
|---|---|
| 120: Transition part | 130: Body part |

A: Occlusion part of blood flow by guiding catheter and tortuous vessel
a: Blood flow entering circulation hole for maintaining blood flow
b: Exit through which blood flow entering circulation hole is discharged
L: Stenosis lesion

### MODE FOR INVENTION

Reference will now be made in detail to the embodiments of the present invention, examples of which are illustrated in the accompanying drawings. However, it will be apparent to those skilled in the art that the following embodiments can be readily understood and modified into various types, and the scope of the present invention is not limited to the embodiments.

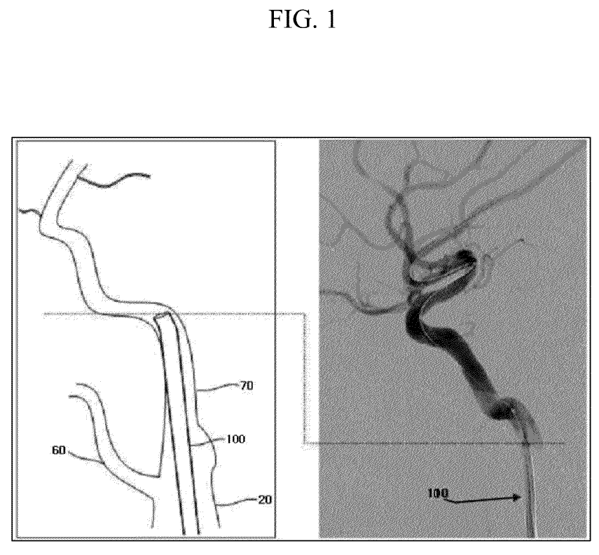

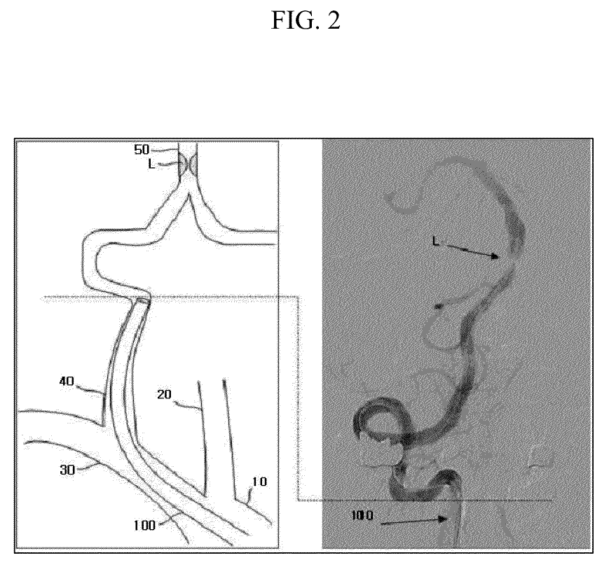

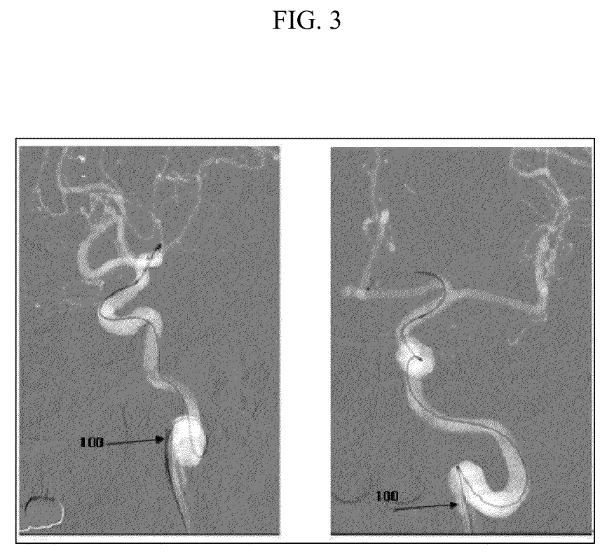

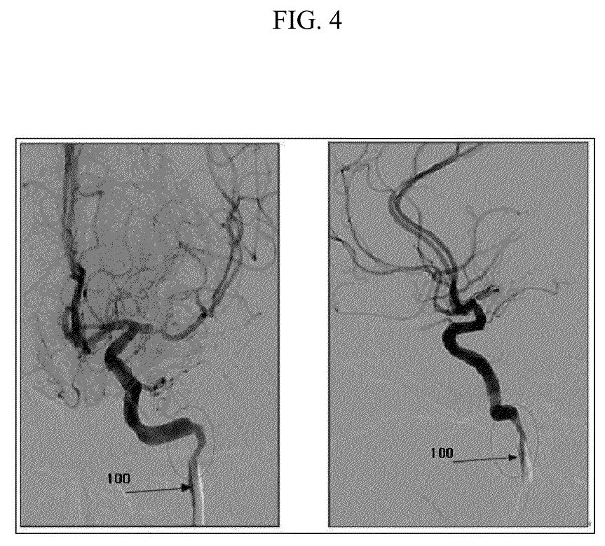

FIG. 1 is a view showing a state in which a conventional guiding catheter is inserted into a vessel of an internal carotid artery 70, and an angiogram thereof, and it will be appreciated that guidance of a distal part is difficult due to a rigid distal end 100, and a vessel is occluded. FIG. 2 is a view showing a state in which the conventional guiding catheter is inserted into a vessel of a vertebral artery 40, and an angiogram thereof, and it will be appreciated that guidance of the distal end is difficult due to the rigid distal end 100, and the vessel is occluded. FIG. 3 is angiograms showing states in which the conventional guiding catheters are inserted into tortuous vessels, and it will be appreciated that guidance of the distal part is difficult due to the rigid distal end 100, and the vessel is occluded. FIG. 4 is angiograms showing states in which spasm (elliptical dotted lines) of vessels are generated by the conventional guiding catheters.

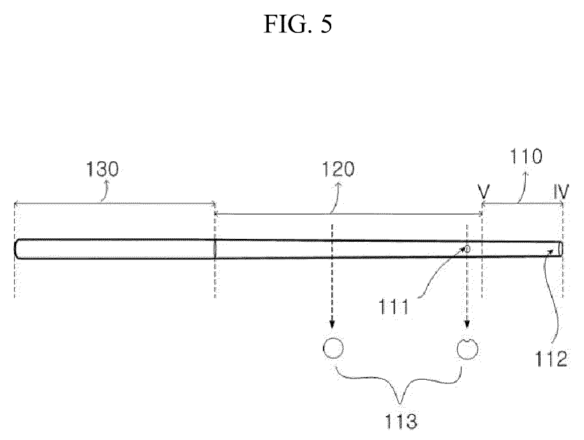

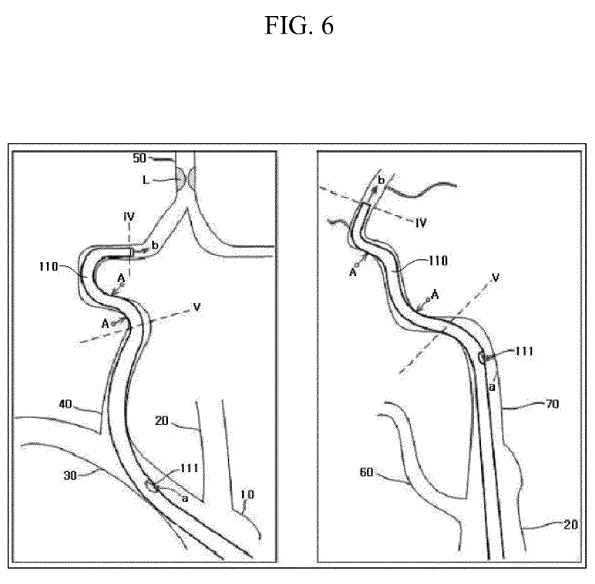

FIG. 5 shows a distal part of a guiding catheter in accordance with an exemplary embodiment of the present invention, and FIG. 6 shows a state in which the distal part of the far-reaching guiding catheter in accordance with the exemplary embodiment of the present invention is inserted into a proximal part of a target vessel for treatment. The guiding catheter in accordance with the present invention has a minimized thickness of a catheter material such that a lumen sufficient to perform an operation in a vessel can be secured, and an outer diameter of the catheter can be minimized. In addition, a blood flow circulation hole 111 is formed in a side surface of the catheter to prevent a blood flow occlusion phenomenon A due to reduction in inner diameter of the vessel itself when the guiding catheter is inserted into the vessel to be treated, securing normal blood flow of the target organ. More specifically, in the guiding catheter in accordance with the present invention in FIG. 6, since a distal part 110 formed of a flexible material can maximally approach the target vessel having a stenosis lesion L, and blood can flow from a to b through the blood flow circulation hole 111 formed in the side surface of the catheter, even when the tortuous vessel has a blood flow occlusion part A, smooth flow of the blood can be maintained.

The guiding catheter in accordance with the exemplary embodiment of the present invention has a large inner diameter such that various operations in the vessel can be easily performed, and the catheter can be inserted into the closest portion of the target vessel when an intervention in the vessel is performed.

Hereinafter, a specification of an embodiment of the far-reaching guiding catheter in accordance with the present invention will be described with reference to FIG. 5. The specification merely exemplarily shows the embodiment of the present invention, and the scope of the present invention is not limited thereto.

A body part 130 of the catheter is formed of a hard material, and has characteristics of the guiding catheter (a proximal part: 40 to 50 cm).

The distal part 110 of the catheter is formed of a very flexible and soft material (the distal part: 15 to 20 cm).

A transition part 120 between the body part and the distal part is formed of a material more flexible than the body part, and less flexible than the distal part (an intermediate part: 45 to 55 cm).

A circulation hole 111 for maintaining blood flow is formed at a portion between 5 and 20 cm from an end of the distal part.

The guide catheter in accordance with the exemplary embodiment of the present invention has an inner diameter between 0.056 and 0.072 inches.

The circulation hole 111 for maintaining a blood flow may be one or more, and the size and shape thereof are not particularly limited as long as blood flow can be maintained. Preferably, a diameter thereof may be between 0.5 and 1 mm.

Major characteristics of the guiding catheter in accordance with the exemplary embodiment of the present invention are as follows: since flexibility of a certain segment of the distal part is maximally increased, and friction is minimized to provide an anatomically tortuous structure, the catheter can be guided to a distal part such as a distal part of an internal carotid artery or a vertebral artery of a C1-2 segment part. In addition, it is possible to prevent damage to a vessel wall or friction with the vessel wall when a surgical material passes through a vessel portion in which the guiding catheter is not inserted, depending on the surgical material.

This invention may be embodied in different forms and should not be construed as limited to the embodiments set forth herein. Rather, these embodiments are provided so that this disclosure will be thorough and complete, and will fully convey the scope of the invention to those skilled in the art. As described above, although the preferable embodiments of the present invention have been shown and described, it will be appreciated by those skilled in the art that substitutions, modifications and variations may be made in these embodiments without departing from the principles and spirit of the general inventive concept, the scope of which is defined in the appended claims and their equivalents.

### INDUSTRIAL APPLICABILITY

As can be seen from the foregoing, a guiding catheter of the present invention can be used in various vascular interventions that are performed through a body passage, and in particular, in a cerebrovascular intervention in which a finer surgery to the distal part is needed. The guiding catheter may be inserted into the closest part of a target vessel to easily perform various operations in a vessel. And, the guiding catheter includes a circular hole for blood circulation to secure normal blood flow of a target organ when a blood flow occlusion phenomenon due to reduction in inner diameter of the vessel due to the distal part of the catheter occurs.

1. A guiding catheter comprising:
a blood flow circulation hole formed in a side surface of the guiding catheter.

2. The guiding catheter, according to claim 1, wherein the guiding catheter has a minimized thickness such that a lumen sufficient for an operation in a vessel is provided and an outer diameter thereof is minimized.

3. The guiding catheter according to claim 1, wherein the guiding catheter has an inner diameter between 0.056 and 0.072 inches.

4. The guiding catheter according to claim 1, wherein the blood flow circulation hole has a diameter between 0.5 and 1 mm.

5. The guiding catheter according to claim 1, wherein the guiding catheter comprises:
a body part formed of a hard material;
a distal part formed of a flexible and soft material; and
a transition part formed of a material more flexible than the body part, and less flexible than the distal part.

6. The guiding catheter according to claim 5, wherein the body part has a length between 40 and 50 cm.

7. The guiding catheter according to claim 5, wherein the distal part has a length between 15 and 20 cm.

8. The guiding catheter according to claim 5, wherein the transition part has a length between 45 and 55 cm.
